(19) United States
(12) Patent Application Publication
(10) Pub. No.: US 2026/0199014 A1
(43) Pub. Date: Jul. 16, 2026

(54) EXTENDED REALITY REGISTRATION METHOD USING VIRTUAL FIDUCIALS

(71) Applicant: Xironetic LLC, Oklahoma City, OK (US)

(72) Inventor: Jeffrey POTTS, Edmond, OK (US)

(21) Appl. No.: 19/132,762

(22) PCT Filed: Nov. 22, 2023
(86) PCT No.: PCT/US2023/080865

§ 371 Date: May 23, 2025

Related U.S. Application Data
(60) Provisional application No. 63/384,777, filed on Nov. 23, 2022.

(51) Int. Cl. A61B 34/10; A61B 17/00; A61B 90/00; A61B 90/94; G06T 19/00; G06V 20/20

(52) U.S. Cl. CPC A61B 34/10; A61B 90/361; A61B 90/39; A61B 90/94; G06T 19/006; G06V 20/20; A61B 2017/00203; A61B 2017/00207; A61B 2017/00216; A61B 2034/105; A61B 2090/365; A61B 2090/3983

(57) ABSTRACT

Disclosed herein are systems and method for placement of a virtual medical overlay on a physical body part using virtual fiducial markers. A method may retrieve a three-dimensional virtual model tagged with a first plurality of virtual fiducial markers each indicative of a respective anatomical landmark of a body part. The method may capture at least one image of the body part in a physical world. In response to receiving a selection of a second plurality of virtual fiducial markers on the at least one image, the method may generate a third plurality of virtual fiducial markers with corrected positions of the second plurality of virtual fiducial markers. The method may generate a virtual overlay of the three-dimensional virtual model on the body part captured by the extended reality device in real-time by performing registration using the third plurality of virtual fiducial markers.

### CROSS-REFERENCE TO RELATED APPLICATIONS

This application claims the benefit of U.S. Provisional Application No. 63/384,777, filed Nov. 22, 2022, which is herein incorporated by reference.

### FIELD OF TECHNOLOGY

The present disclosure relates to the field of extended reality (e.g., virtual reality, augmented reality, mixed reality), and, more specifically, to systems and methods for placement of a virtual medical overlay on a physical body part using virtual fiducial markers.

### BACKGROUND

Many applications of extended reality in medicine seek to establish correspondence of three-dimensional patient data (e.g., a visual overlay) with the physical patient in a step known as registration. Registration of virtual digital data with the anatomy of a patient is a critical step in surgical extended reality applications and requires accurate placement of three-dimensional models, which may be constructed from, for example, preoperative CT and MRI data.

Currently, imaging systems use optical trackers or computer vision tools to perform registration. Computer vision tools commonly utilize custom fiducial markers. The placement of the fiducial markers dictates the accuracy of the registration. For example, poorly placed markers will result in a misaligned visual overlay.

Intraoperatively, fiducial markers may become dislodged or may register erroneous spatial coordinates for various reasons (e.g., saturation of tracking cameras due to intense surgical lighting, contamination of fiducial markers with blood, etc.). It may become necessary for a surgeon to adjust the spatial coordinates of fiducial markers during the course of surgery. This may involve significant disruption to the surgical workflow, as well as activities that may jeopardize sterility of a surgical field, such as the surgeon re-adjusting fiducial markers, or manually adjusting spatial coordinates of virtual fiducial markers.

To ensure the accuracy of marker placement, certain types of optical tracking technology and marker-based tracking technologies require a special-purpose radiologic imaging protocol pre-operatively to register fiducial markers with the anatomy of a patient. However, the protocol involves increased radiation exposure to patients as well as increased costs.

Conventional imaging systems utilize morphometric measurements with point clouds or neural network-based image analytics to perform registration. However, these approaches are often limited in scope of application due to insufficient training data needed to accommodate the anatomical variation of different patients. Conventional imaging systems have also disclosed methods for anatomical landmark selection to assist in the registration of 3D imaging data. However, such methods utilize tools or other instruments (e.g., lasers) to indicate a location without providing a visual feedback mechanism to the user of the selected location. Such methods also offer limited means to edit the location, and no means to verify the correct identification of a set of landmarks. All of these shortcomings limit the effectiveness of a landmark-based registration approach.

### SUMMARY

In one exemplary aspect, the techniques described herein relate to a method for placement of a virtual medical overlay on a physical body part using virtual fiducial markers, the method including: retrieving, from memory by an extended reality device, a three-dimensional virtual model of a body part, wherein the three-dimensional virtual model is tagged with a first plurality of virtual fiducial markers each indicative of a respective anatomical landmark on the body part; capturing, by the extended reality device, at least one image of the body part in a physical world; generating a request to mark, using the at least one image, a second plurality of virtual fiducial markers corresponding to anatomical landmarks indicated by the first plurality of virtual fiducial markers; in response to receiving a selection of the second plurality of virtual fiducial markers based on the request, generating a third plurality of virtual fiducial markers with corrected positions of the second plurality of virtual fiducial markers; and generating a virtual overlay of the three-dimensional virtual model on the body part captured by the extended reality device in real-time by performing registration on the first plurality of virtual fiducial markers and the third plurality of virtual fiducial markers.

In some aspects, the techniques described herein relate to a method, wherein generating the third plurality of virtual fiducial markers includes, for each respective marker of the second plurality of virtual fiducial markers: identifying, within the at least one image, a region associated with the respective marker, wherein the respective marker is represented by coordinates and the region is bounded by a threshold distance from the coordinates; fitting, within the region, at least one polynomial curve on a visual feature captured in the at least one image; retrieving, from the memory, polynomial constraints associated with an anatomical landmark of the respective marker and a relative position of a corrected marker based on the polynomial constraints; and aligning the respective marker with the corrected marker by comparing the at least one polynomial curve against the polynomial constraints, wherein the aligned respective marker is included in the third plurality of virtual fiducial markers.

In some aspects, the techniques described herein relate to a method, wherein generating the third plurality of virtual fiducial markers includes: identifying a first anchor marker in the first plurality of virtual fiducial markers; generating a first plurality of vectors originating from the first anchor marker to remaining markers in the first plurality of virtual fiducial markers; identifying a second anchor marker in the second plurality of virtual fiducial markers; generating a second plurality of vectors originating from the second anchor marker to remaining markers in the second plurality of virtual fiducial markers; and repositioning each of the second plurality of virtual fiducial markers such that a difference between the first plurality of vectors and the second plurality of vectors is less than a threshold difference, wherein repositioned markers in the second plurality of virtual fiducial markers are included in the third plurality of virtual fiducial markers.

In some aspects, the techniques described herein relate to a method, wherein generating the third plurality of virtual fiducial markers includes: generating a virtual mesh of body part using the at least one image and sensors of the extended reality device, wherein the virtual mesh includes depth information of the body part; and for each respective marker of the second plurality of virtual fiducial markers: determining a terminus point of a virtual ray used to select the respective marker, wherein the terminus point is an intersection of the virtual ray and the virtual mesh; and repositioning the respective marker along a depth axis based on the terminus point, wherein the respective marker repositioned is included in the third plurality of virtual fiducial markers.

In some aspects, the techniques described herein relate to a method, further including: generating the three-dimensional virtual model of the body part by performing segmentation on one or more of: X-ray scan, MRI scan, CT scan, ultrasound scan.

In some aspects, the techniques described herein relate to a method, further including: tagging the generated three-dimensional virtual model with the first plurality of virtual fiducial markers.

In some aspects, the techniques described herein relate to a method, wherein the second plurality of virtual fiducial markers includes at least four markers and at most ten markers.

In some aspects, the techniques described herein relate to a method, further including: placing a virtual fiducial marker on a physical optical code placed on the body part; and updating positions of the third plurality of virtual fiducial markers in response to detecting a change in a position of the virtual fiducial marker, wherein the virtual fiducial marker is a reference point with a trackable position.

In some aspects, the techniques described herein relate to a method, wherein the extended reality device is worn by a user, and wherein the extended reality device includes at least one camera and a display configured to generate virtual reality visuals, augmented reality visuals, or mixed reality visuals.

In some aspects, the techniques described herein relate to a method, wherein the virtual fiducial selection is received by any combination of: (1) tracking a gaze of the user by the at least one camera; (2) tracking a hand or finger position of the user by the at least one camera; (3) tracking a stylus position by the at least one camera; (4) detecting a voice command by the user; and (5) detecting a physical input on the extended reality device.

In some aspects, the techniques described herein relate to a method, further including predicting positions of each of the third plurality of virtual fiducial markers using at least one machine learning model trained to output the positions based on an input including the at least one image and the three-dimensional virtual model.

In some aspects, the techniques described herein relate to a method, further including: detecting at least one marker outlier based on a comparison between the first plurality of virtual fiducial markers and the second plurality of virtual fiducial markers; and determining a corrected position solely for the at least one marker outlier for inclusion in the third plurality of virtual fiducial markers.

It should be noted that the methods described above may be implemented in a system comprising a hardware processor. Alternatively, the methods may be implemented using computer executable instructions of a non-transitory computer readable medium.

In some aspects, the techniques described herein relate to a system for placement of a virtual medical overlay on a physical body part using virtual fiducial markers, including: at least one memory; and at least one hardware processor of an extended reality device, wherein the at least one hardware processor is coupled with the at least one memory and configured, individually or in combination, to: retrieve, from the at least one memory, a three-dimensional virtual model of a body part, wherein the three-dimensional virtual model is tagged with a first plurality of virtual fiducial markers each indicative of a respective set of anatomical landmarks on the body part; capture at least one image of the body part in a physical world; generate a request to mark, using the at least one image, a second plurality of virtual fiducial markers corresponding to anatomical landmarks indicated by the first plurality of virtual fiducial markers; in response to receiving a selection of the second plurality of virtual fiducial markers based on the request, generate a third plurality of virtual fiducial markers with corrected positions of the second plurality of virtual fiducial markers; and generate a virtual overlay of the three-dimensional virtual model on the body part captured by the extended reality device in real-time by performing registration on the first plurality of virtual fiducial markers and the third plurality of virtual fiducial markers.

In some aspects, the techniques described herein relate to a non-transitory computer readable medium storing thereon computer executable instructions for placement of a virtual medical overlay on a physical body part using virtual fiducial markers, including instructions for: retrieving, from memory by an extended reality device, a three-dimensional virtual model of a body part, wherein the three-dimensional virtual model is tagged with a first plurality of virtual fiducial markers each indicative of a respective anatomical landmark on the body part; capturing, by the extended reality device, at least one image of the body part in a physical world; generating a request to mark, using the at least one image, a second plurality of virtual fiducial markers corresponding to anatomical landmarks indicated by the first plurality of virtual fiducial markers; in response to receiving a selection of the second plurality of virtual fiducial markers based on the request, generating a third plurality of virtual fiducial markers with corrected positions of the second plurality of virtual fiducial markers; and generating a virtual overlay of the three-dimensional virtual model on the body part captured by the extended reality device in real-time by performing registration on the first plurality of virtual fiducial markers and the third plurality of virtual fiducial markers.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is a block diagram illustrating a system for placement of a virtual medical overlay on a physical body part using virtual fiducial markers.

FIG. 2 illustrates examples of locations of physical anatomical landmarks on a face of a patient.

FIG. 3 illustrates a diagram for anatomical landmarking with virtual fiducial markers from the perspective of a surgeon.

FIG. 4 illustrates a preparatory process for establishing landmark locations on virtual three-dimensional models of a patient.

FIG. 5 illustrates results of performing registration.

FIG. 6 illustrates the use of a virtual ray concept.

FIG. 7 illustrates the use of surface mesh analysis to refine the depth positions of indicated virtual fiducial markers to improve coincidence with physical anatomical landmarks.

FIG. 8 illustrates the use of image analysis to refine the position of a virtual fiducial marker.

FIG. 9 illustrates the use of shape analysis to evaluate possible outliers in intraoperative virtual fiducial marker placement by comparison with the preoperative fiducial marker indications.

FIG. 10 illustrates the use of a single fiducial marker on a patient for tracking purposes.

FIG. 11 illustrates the use of a stylus probe with an affixed optical marker to place virtual fiducial markers.

FIG. 12A illustrates a flowchart of a method for performing registration using virtual fiducial markers.

FIG. 12B illustrates a flowchart of a method for performing registration using virtual fiducial markers while showing tasks required intraoperatively by the surgeon to perform registration of three-dimensional data on the patient.

FIG. 12C illustrates components of the virtual fiducial marker refinement process.

FIG. 13 illustrates an example graphical user interface of a software for landmark placement.

FIG. 14 is a flow diagram illustrating a method for placement of a virtual medical overlay on a physical body part using virtual fiducial markers.

FIG. 15 presents an example of a general-purpose computer system on which aspects of the present disclosure may be implemented.

### DETAILED DESCRIPTION

Exemplary aspects are described herein in the context of a system, method, and computer program product for placement of a virtual medical overlay on a physical body part using virtual fiducial markers. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the example aspects as illustrated in the accompanying drawings. The same reference indicators will be used to the extent possible throughout the drawings and the following description to refer to the same or like items.

As described in the background, there are many shortcomings of conventional imaging systems. There is a need for efficiently and cost-effectively improving the placement of fiducial markers for proper placement of a medical overlay (used interchangeably with visual overlay). Methods that can accurately and quickly register data to the patient without the downsides of multiple custom physical fiducial markers and specialized clamps may also allow for fewer distractions to the surgeon, and therefore less risk to the patient.

The present disclosure describes systems and methods for the placement of three-dimensional medical data for visualization over a physical body part during a medical operation or surgical procedure using extended reality, which is a general term for technologies such as augmented, mixed, and virtual reality. In extended reality, digital information is overlaid on the physical world of the observer. Relative to the present disclosure, the digital information may include three-dimensional data such as models of internal anatomy belonging to a patient. For example, such models may be used by a surgeon as guides during an operation.

It should be noted that the systems and methods of registration described allow for, but do not require, physical fiducial marker tracking markers and do not require a special-purpose pre-operative radiologic imaging test (thereby decreasing radiation exposure to the patient). The systems and methods minimize interference with the surgical workflow, and minimize risk of breach of sterility during surgery. The systems and methods further allow for flexibility to the surgeon based on intraoperative decision-making and contingency planning.

In some aspects, the systems and methods may leverage existing knowledge of anatomical landmarks as well as current augmented reality hardware (e.g., the Microsoft HoloLens 2™ head-mounted display (HMD)). Landmarks are anatomical features that are easily identified and reproducible among examiners. A significant volume of published research confirms accuracy and reproducibility of anatomical landmarks during repeat measurements and in-between examiners, confirming inter-observer and intra-observer reliability.

In one aspect, the systems and methods work by associating pre-defined landmarks on the virtual data with virtual fiducial markers placed by the surgeon on the physical patient. Preoperatively, a set of landmarks are defined by the surgeon that will use the extended reality device to aid them in surgery. These landmarks are indicated in a preoperative planning software that runs on a computer separate to the extended reality device, and are placed using software on the three-dimensional patient models created by segmentation of preoperative scan data such as computed tomography (CT) and magnetic resonance imaging (MRI) scan data. Once selected, these landmarks are stored in computer memory and associated with a set of coordinates in the same coordinate space as the patient three-dimensional data. This data is then loaded into the extended reality device as part of an application accessed during the surgical procedure.

In the operating room, the surgeon places virtual fiducial markers on some or all of the predefined anatomical landmark locations on the physical patient, using either a gesture-based or gaze-based process on the extended reality device. Mesh surface maps of the patient, image analysis, and shape analysis allow for refinement to initial virtual fiducial marker indications by the surgeon. Virtual fiducial marker placement may be performed using virtual or physical indicators, and positions are editable after initial placement. Once landmark locations are set, the surgeon instructs the software on the extended reality device to perform registration using algorithms known in the art to minimize an error function when aligning two sets of coordinates.

Once completed, the three-dimensional data (e.g., the visual overlay) moves with the patient by tracking the location of the landmark points using image processing. Optionally, the surgeon may use optical code(s) on the patient if advantageous for tracking for that particular surgical case. By using this process, the software is able to position three-dimensional holographic overlays on the patient with millimeter accuracy, without requiring the use of intraoperative radiation or specialized markers during preoperative imaging, and with the ability to accommodate a wide variety of scenarios intraoperatively with regards to patient positioning and draping.

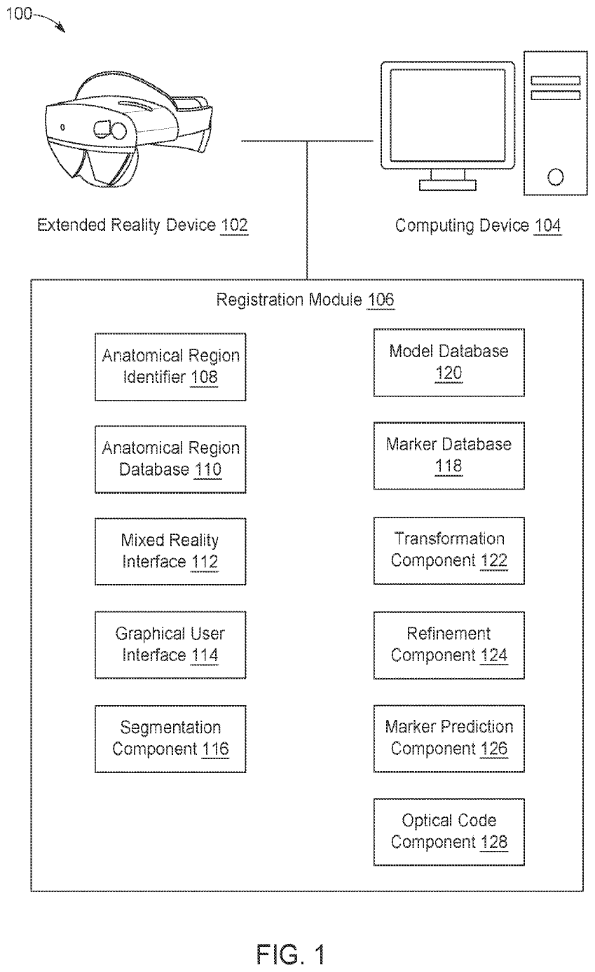

FIG. 1 is a block diagram illustrating system 100 for placement of a virtual medical overlay on a physical body part using virtual fiducial markers. System 100 includes registration module 106, which may be a software application that is installed on extended reality device 102 and on computing device 104. In one aspect, extended reality device 102 is a wearable device that includes a display and at least one camera. A camera may be any type of imaging device, including but not limited to an optical camera, an infrared camera, a structured light camera, or a time-of-flight sensor. In particular, extended reality device 102 may include augmented reality, virtual reality, and mixed reality features that enable a user to view a physical scene via a display while simultaneously viewing a virtual overlay. The at least one camera may be used to capture the physical scene and/or track the eyes of the wearer. In some aspects, extended reality device 102 is a head-mounted display such as the Microsoft HoloLens 2™. In other aspects, extended reality device 102 is lightweight eyewear (e.g., smart glasses).

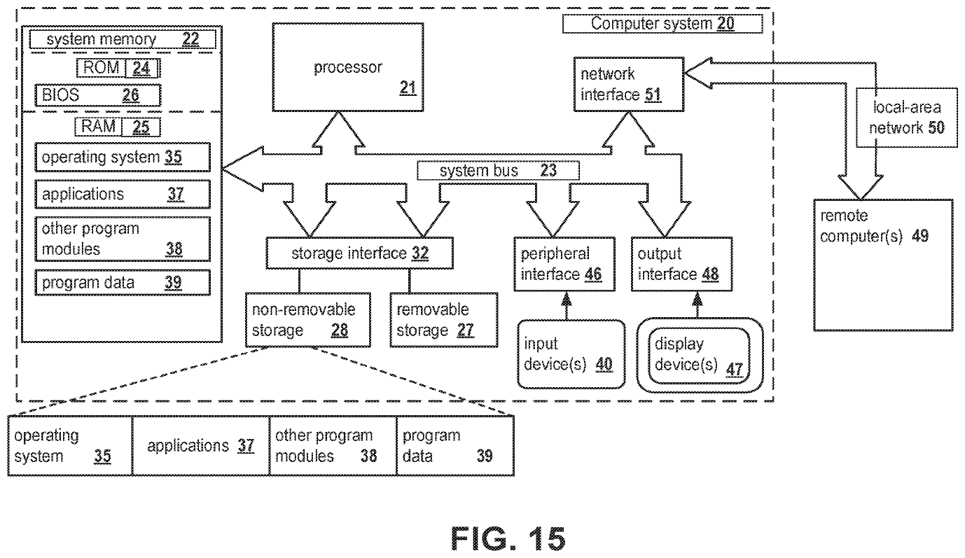

Computing device 104 is a computer system (described in FIG. 15) that is configured to communicate with extended reality device 102. The respective devices may exchange data (e.g., virtual fiducial markers, images, videos, clickstream data, etc.) intraoperatively and/or preoperatively using wireless communication links such as Wi-Fi, Bluetooth, etc., and/or wired communication links such as USB cables, Ethernet cables, etc. Each of the devices 102 and 104 may include one or more hardware processors and memories. It should be noted that any actions performed (e.g., computations, data processing, imaging, etc.) are performed by the one or more hardware processors and any data stored or retrieved, unless otherwise indicated, is in the memories.

Registration module 106 may include two graphical user interfaces depending on the device executing the application. For example, registration module 106 may have a mixed reality interface 112 when executed on extended reality device 102, and may have a standard graphical user interface (GUI) 114 when executed on computing device 104 (e.g., a desktop computer, a laptop, a tablet, etc.).

From a high level, registration module 106 establishes correspondence between two sets of coordinates, which are defined by virtual fiducial markers. One set of coordinates is defined preoperatively on computing device 104 using the standard GUI 114 of registration module 106 where the user places virtual fiducial markers (e.g., using a touchscreen, mouse, keyboard, etc., connected to computing device 104) or through use of automated or semi-automated techniques for landmark detection. The virtual fiducial marker coordinates for the preoperative landmarks are preserved in the coordinate frame of the 3D model(s) to be viewed on the extended reality device 102 via mixed reality interface 112. For example, if a CT-scan is performed of the head of a patient, the first set of coordinates is selected on a 3D model generated using the CT-scan.

The second set of coordinates is defined intraoperatively by the surgeon who indicates (e.g., using gesture and/or gaze-based interactions, voice commands, etc.) the placement of virtual fiducial markers relative to the coordinate frame of the physical patient. Once a complete set of coordinates for both sets of virtual fiducial markers is defined, registration module 106 computes a transformation on the 3D model to minimize an error function, such as a calculation of least-squares error between the two sets of coordinates. The result of applying such a transformation is that the 3D model(s) of the patient anatomy is accurately registered onto the physical patient.

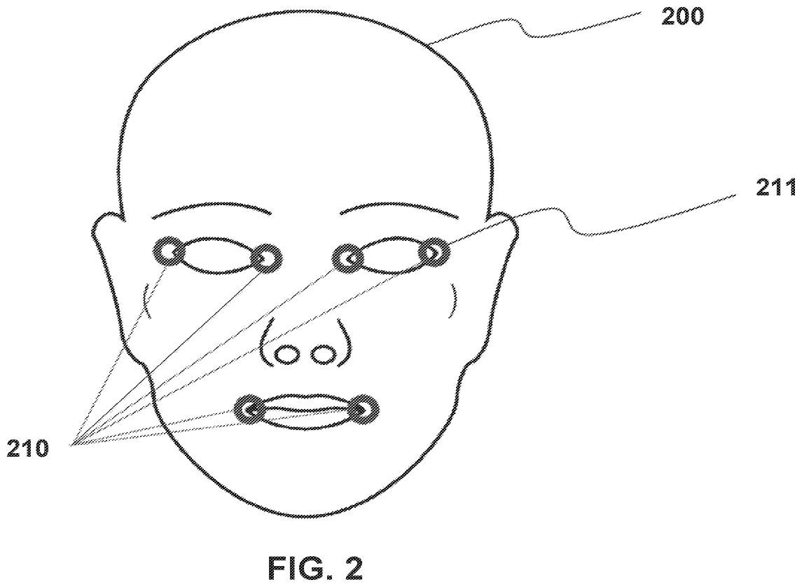

FIG. 2 illustrates the concept of physical anatomical landmarks on the face of a patient. Patient 200 has identifiable physical anatomical landmarks 210 on each corner of the eyes, the tip and base of the nose, and the edges of the lips. The surgeon can unambiguously identify these landmarks with anatomical references, for example, point 211 at the outer edge of the left eye, the left lateral canthus, will generally be clearly identifiable by the surgeon while wearing extended reality device 102.

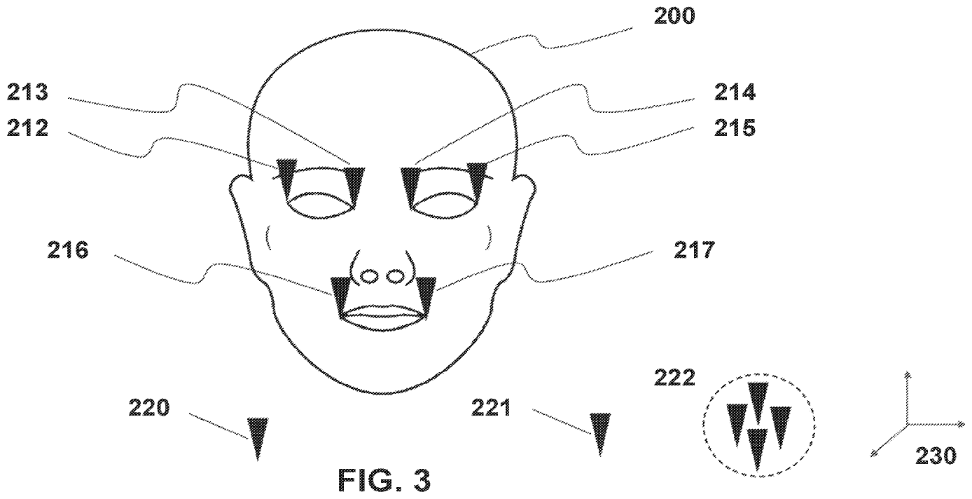

FIG. 3 illustrates the concept of anatomical landmarking with virtual fiducial markers, from the point of view of the surgeon looking at the patient 200 through extended reality device 102. The example shown in FIG. 3 is just for illustration, and it should be understood that this same technique may be applied to a wide variety of patient anatomical targets, with varying amounts of virtual fiducial markers.

As discussed before, registration module 106 on extended reality device 102 allows the surgeon to view both the patient and digital information simultaneously. Virtual fiducial markers 220 may be 2D or 3D objects of different shapes and sizes to provide visual indication to the surgeon of their placement on the mixed reality interface. In FIG. 3, the virtual fiducial markers are illustrated as triangular or conic objects, with their tip pointing to the precise location of the 3D coordinates defined for that particular landmark. All virtual fiducial marker coordinates are defined in coordinate frame 230 that is referenced to the physical environment that the extended reality device 102 application is being used in, for example, an operating room.

For example, in reference to FIG. 3, registration module 106 may receive an image of the view in front of the wearer (as captured by a camera of extended reality device 102). In one aspect, registration module 106 may execute anatomical region identifier 108, which is an object detection classifier (e.g., a Bayes classification machine learning algorithm) configured to identify the anatomical region in the image. In another aspect, anatomical region identifier 108 may generate a virtual menu on the mixed reality interface 112 and request that the wearer select an anatomical region from a plurality of anatomical regions on the virtual menu.

Based on the identified/selected anatomical region, registration module 106 retrieves a plurality of landmark requests associated with the anatomical region from anatomical region database 110 (stored in the memory of extended reality device 102 and/or computing device 104). Each set of landmark requests may be specific to the anatomical region. For example, landmarks needed for a hand may be different than landmarks needed for a face. Registration module 106 subsequently generates, on extended reality device 102, the plurality of requests to locate the necessary landmarks. The requests may be visual or audio prompts that guide a surgeon through the registration process and enable registration module 106 to receive coordinates of said landmarks.

Referring to FIG. 3, anatomical region identifier 108 may determine that the anatomical region being viewed is the face of the patient. Registration module 106 searches for the landmarks associated with the face in anatomical region database 110 (which may include a table that lists landmarks for various anatomical regions) and generates, one landmark at a time, a plurality of landmark requests. For example, registration module 106 may generate a request for the surgeon to locate the first landmark, 212, which is the right corner of the right eye-the right lateral canthus. In some aspects, a request is generated as hovering text on the mixed reality interface 112 prompting the wearer (i.e., the surgeon) to make a selection. In response, registration module 106 receives a selection of the requested landmark via mixed reality interface 112. The selection includes coordinates in coordinate frame 230. Accordingly virtual fiducial marker 220 is placed using techniques defined herein on the physical patient's right corner of their right eye.

In some aspects, extended reality device 102 includes an external camera that captures images of the view in front of the surgeon. Using the external camera, extended reality device 102 may execute hand tracking algorithms to receive selections. For example, the surgeon may make a point gesture at a particular point and extended reality device 102 may interpret the tip of the index finger as pointing to the selected coordinates. In some aspects, extended reality device 102 includes an internal camera that captures images of the eyes of the surgeon. Using the internal camera, extended reality device 102 may execute gaze tracking algorithms to receive selections. For example, the surgeon may stare at a particular point on the patient and perform a long blink (e.g., lasting 3 seconds or more) or consecutive blinks (e.g., three blinks in 1 second) to indicate a selection. In some aspects, extended reality device 102 includes a microphone that captures audio clips. Using the microphone, extended reality device 102 may execute voice commands. For example, extended reality device 102 may receive selections using a combination of gaze, hand motion, and voice commands (e.g., the surgeon may stare or point at a location and say “capture”).

Registration module 106 then generates a request to locate the second landmark 213, which is the left corner of the patient's right eye. A second virtual fiducial marker 221 is then placed based on the received selection from the surgeon by the same method(s) used to place virtual fiducial marker 220. Two additional landmarks on corners of the patient's left eye, 214 and 215, are also shown in FIG. 3. Two additional landmarks 216 and 217 for the corners of the lips are illustrated in FIG. 3. The remaining set of virtual fiducial markers 222 are placed until the minimum number of intraoperative virtual fiducial markers required for the particular surgical case have been placed. There need not be a one-to-one correspondence in the number of preoperative virtual fiducial markers and intraoperatively indicated fiducial markers, but the user must place at least a threshold number (e.g., four) of intraoperative fiducial markers. Preferably at least six markers are requested by registration module 106.

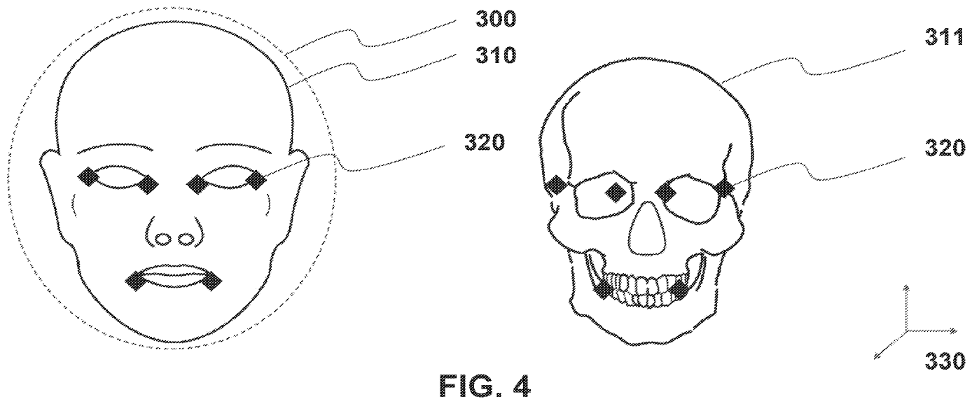

FIG. 4 illustrates the preoperative planning process required to define the virtual fiducial markers on the 3D data. In an exemplary aspect, registration module 106 segments (e.g., by segmentation component 116) raw data from CT and/or MRI scans into 3D model(s) represented as triangular meshes that closely approximate the 3D geometry of anatomical structures, such as bone, muscle, tissue, organs, etc. The preoperative virtual fiducial markers defined by this process are placed by registration module 106 relative to a group of 3D models 300 which may include a variety of anatomical structures, including a 3D model 310 of the patient's skin and a 3D model 311 of the patient's skull. The grouping of 3D models 300 have the 3D positions of all associated anatomical structures such as skin model 310 and skull model 311 preserved with the scale and spacing of the physical patient the data was collected from. In some aspects, registration module 106 may further store the 3D models in model database 120.

Using graphical user interface 114 on computing device 104, a surgeon, imaging specialist, or other technologist can place virtual fiducial markers by viewing the models on a monitor. The virtual fiducial markers 320 may be 2D or 3D objects of different shapes and sizes to provide visual indication to the user of their placement.

The virtual fiducial markers 320 may be selected based on the requirements for that particular surgical case. If it is known that, for example, the eyes and lips will be uncovered for part of the surgical procedure, then virtual fiducial marker locations as shown in FIG. 4 could be good candidate locations for the case. However, in a preferred aspect, a large selection of preoperative virtual fiducial markers is indicated, so that the surgeon has flexibility during the surgical case to use only a subset of all available landmarks to perform the registration process.

Subsequent to receiving indications of the placement of the set of virtual fiducial markers 320 relative to the 3D models 300, registration module 106 saves the locations of the virtual fiducial markers 320 in marker database 118 by exporting their three-dimensional coordinates in the 3D model coordinate frame 330. The coordinates may be an ordered pair of decimal numbers stored as a vector but may take other representations as advantageous to the method. Marker database 118 may store a collection of marker coordinates for various patients and may used as later reference for the surgeon and/or developer of registration module 106.

After the coordinates of the virtual fiducial markers 320 have been defined, on computing device 104 exports the desired 3D models for display on the extended reality device 102 via mixed reality interface 112. All of the 3D models in group 300 may not be desired to be viewed by the surgeon during the operation; for example, skin model 310 may not be desired for use on the extended reality device 102. While the placement of virtual fiducial markers 320 is most effective relative to skin model 310, the skin model 310 may be discarded following virtual fiducial marker placement or simply left stored in model database 120. The placement of virtual fiducial markers 320 does not change regardless of the models removed from group 300, because the locations of the virtual fiducial markers is made relative to a 3D model coordinate frame 230.

In some aspects, a 3D model and/or virtual fiducial markers on a 3D model may be modified on computing device 104 and sent to extended reality device 102 during the registration process. In this case, the 3D model being used on extended reality device 102 is updated in real-time to the modified 3D model.

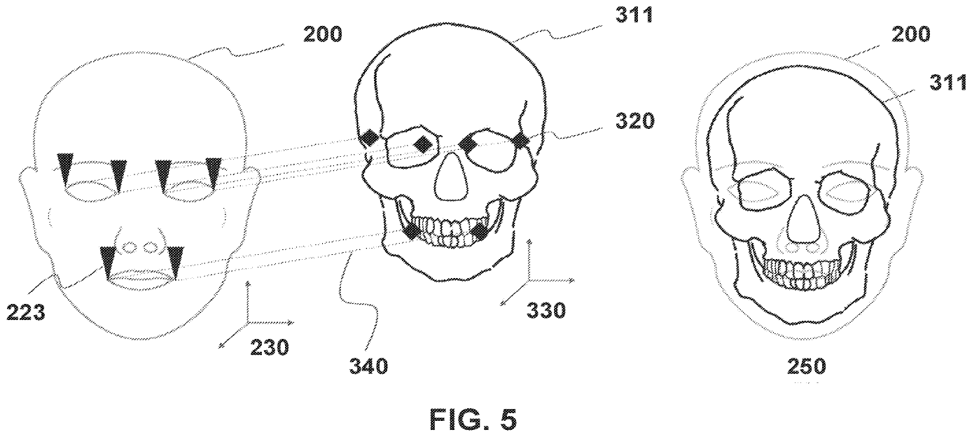

FIG. 5 illustrates the principle of registration by performing a coordinate transformation 340 between the set of virtual fiducial markers 223 and 320. The set of virtual fiducial markers 320 are defined as illustrated in FIG. 4, placed according to the 3D models of segmented CT and/or MRI scan data, relative to coordinate frame 330, based on the knowledge of the surgical plan and availability of the patient's anatomical landmarks during surgery. The set of virtual fiducial markers 223 are defined as illustrated in FIG. 3, placed according to the predefined anatomical landmarks on the physical patient and relative to coordinate frame 230.

In some aspects, the coordinate transformation 340 executed by transformation component 122 involves minimizing the least-squares error between the sets of coordinates, represented in one aspect as 3-dimensional vectors, such that a 3D model(s) such as 311 is registered to the physical patient 200. Therefore, the set of virtual fiducial marker coordinates in coordinate frame 330 are transformed to have one-to-one correspondence with the virtual fiducial marker coordinates in coordinate frame 230, which is defined relative to the physical environment such as the operating room.

The coordinate transformation 340 results in the registration of 3D model(s) 311, which may not be physically visible to the surgeon without extended reality device 102, in the correct spatial position. Therefore, the result of coordinate transformation 340 is to produce a registered view 250 of the 3D models 311 on the physical patient 200 with a high degree of accuracy.

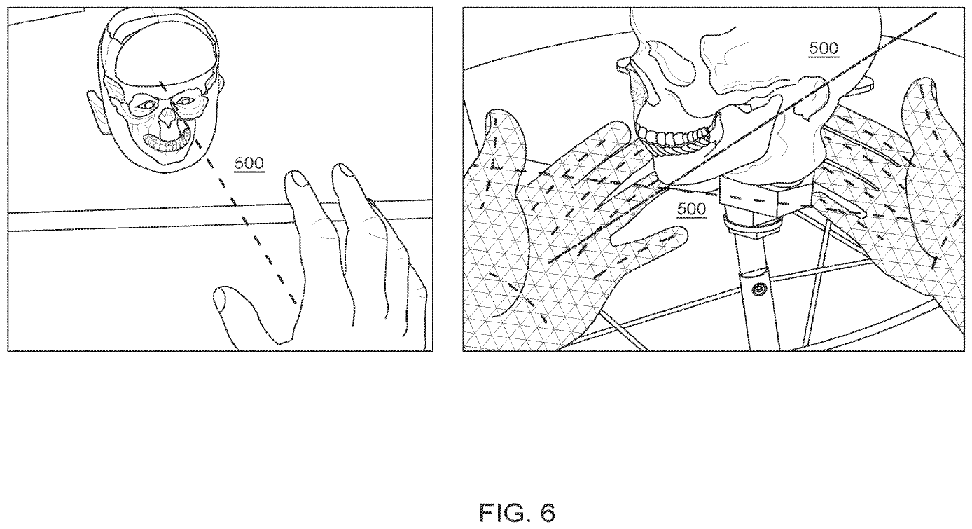

FIG. 6 illustrates the concept of a virtual ray 500. By way of example, extended reality device 102 (e.g., Microsoft HoloLens 2TM) may natively perform hand tracking using an array of onboard sensors including greyscale cameras, depth cameras, and IR cameras. This hand tracking feature allows for virtual rays to be created for interacting with and manipulating virtual objects. For instance, a virtual ray may be a straight line emanating from the center of the user's palm and normal to the back of their hand. A virtual ray may emanate from both hands and may be configured to orient in different directions.

FIG. 6 shows examples of virtual rays 500 from the perspective of the extended reality device 102 wearer. Alternatively, virtual ray 500 may emanate from the extended reality device 102 wearer's head, such as a location between the eyes near the nasion. The concept of the virtual ray enables accurate placement, and its movement may be filtered with techniques known in the art such as a Kalman filter to stabilize its movement against a person's natural fine movement and jitter. Alternatively, a physical object such as the user's finger or a stylus probe of known geometry may be used to indicate the position of virtual fiducial markers relative to the physical patient as described herein. FIG. 6 is not intended to be limiting in any way on the implementation of the virtual ray and illustrates only one possible aspect.

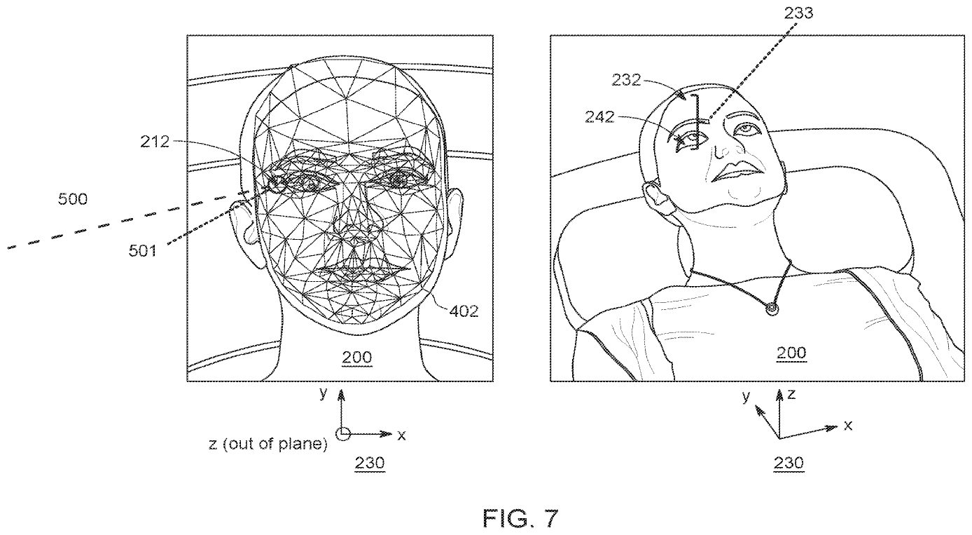

FIG. 7 illustrates the first refinement step which enables the virtual fiducial marker method to improve accuracy of placement relative to anatomical landmarks beyond methods currently known in the art. From the extended reality device 102, once the surgeon instructs the software to perform registration, a virtual ray 500 is projected from either the surgeon's hand or head as described above. The virtual ray appears as a dashed line and has a terminus 501 at a physical surface that the extended reality device 102 has meshed to determine its surroundings. The terminus 501 of virtual ray 500 is inconsistently visible to the extended reality device 102 wearer if directed at a non-planar object in a room, such as a person's face. In many cases, the extended reality device 102's sensors will attempt to ignore subjects in view such as a person, opting to focus on stable spatial anchors present in a room.

Continuing reference to FIG. 7, registration module 106 may generate a dense virtual mesh representation that conforms to the patient's skin. This may be done using the same physical sensor array used natively by extended reality device 102 to create a map of the room and major stationary objects that are controlled by a novel set of algorithms, which instruct the extended reality device 102 to form a dense mesh contouring to the surface in the area that the virtual ray is projected. That is, the surgeon can aim a virtual ray 500 at the surface of the physical patient 200 to instruct the application to attend to an object that may not be completely stationary (such as a patient) to create a virtual mesh of the surface of patient's skin that conforms to the geometry of the patient. This mesh 402 can provide an accurate approximation of the patient's skin contours which is then used to inform virtual fiducial marker placement. Proposals for virtual mesh 402 can come from one or more embedded extended reality device 102 sensors as well as external cameras or sensors, and established through algorithms known in the art to align to the patient's face.

In one aspect, registration module 106 may use virtual mesh 402 to propose virtual fiducial markers 212 directly without the need for the surgeon to indicate landmark locations on the patient. However, virtual mesh 402 is principally used for virtual fiducial marker location refinement, as the virtual mesh may not always accurately place virtual fiducial markers due to the wide variation in human anatomy.

FIG. 7 further illustrates the process and the benefits of using virtual mesh 402 to refine placement of virtual fiducial markers. When the surgeon selects coordinates the placement of virtual fiducial marker 212 on an anatomical landmark location of the physical patient 200, the placement occurs at or very close to the location of the virtual ray terminus 501 (if it exists). With virtual mesh 402 present, virtual ray terminus 501 will exist over a large region of interest on the patient's skin as it contours the virtual mesh, following the intersection of virtual ray 500 with the virtual mesh 402. When placing virtual fiducial marker 212, if not using a virtual mesh 402, it is probable that the coordinates of the virtual fiducial marker are projected away from (either in front of or behind) the normal to the surface of the physical patient's face. Specifically, with reference to coordinate system 230 for both images in FIG. 7, placement of virtual fiducial markers by the surgeon may be highly accurate in the x-y plane, but can often contain error in the z-direction. This error along the z-direction, commonly called depth error as it occurs normal to the patient's skin, is represented by 233 and may be represented by the norm of the vector from coordinate 232 representing an inaccurate virtual fiducial marker placement, and 242, which represents the ideal location of the virtual fiducial marker corresponding to the anatomical landmark position. Because virtual mesh 402 provides a depth contour of the patient's skin, it allows the surgeon to focus on placing virtual fiducial markers accurately in the x-y plane, with depth error 233 minimized by placing the virtual fiducial marker at the z-coordinate corresponding to the position of virtual ray terminus 501 calculated from the intersection of virtual ray 500 with virtual mesh 402.

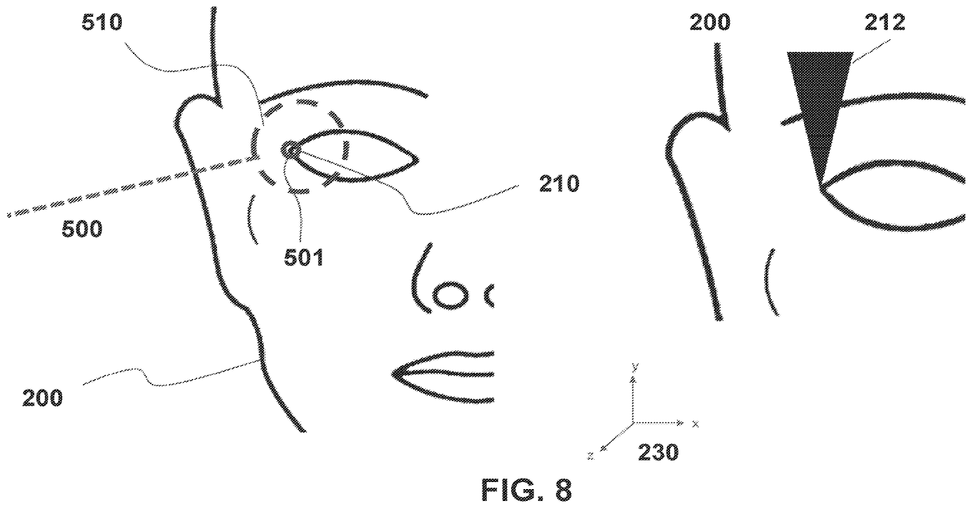

FIG. 8 illustrates a portion of the method to refine to placement of virtual fiducial markers relative to the anatomical landmarks on physical patient 200. In one aspect, registration module 106 may receive a voice command from the surgeon to place the virtual fiducial marker for a particular anatomical landmark. Registration module 106 may activate a camera of extended reality device 102 to take a picture or capture a short video clip from its vantage point. This photo or video clip of the physical patient is sampled in a region 510 around the virtual ray terminus 501 using image processing techniques such as edge detection, line tracing, and curve fitting to refine, by refinement component 124, the placement of virtual fiducial marker 212 to be more accurately positioned relative to the physical anatomical landmark 210. Alternatively, this portion of the method may preemptively suggest a landmark position or region of interest to the surgeon based on image analysis.

For example, refinement component 124 may fit polynomial curves to the eyelid contours within region 510 and use the intersection of the two curves to refine the placement of a virtual fiducial marker in that region. In some aspects, the polynomial curves defining any part of an anatomical region may be stored in anatomical region database 110. This step provides improved lateral positioning, as indicated by the x-y plane of coordinate frame 230, of the virtual fiducial marker 212 relative to taking the exact position of the virtual fiducial marker as placed by the user's gaze or gesture input. For example, the surgeon may provide an initial selection of coordinates where marker 212 should be placed. Refinement component 124 may determine which landmark the selection is associated with (e.g., the right corner of the right eye) and retrieve polynomial curve constraints from anatomical region database 110. Refinement component 124 may further fit polynomial curves (e.g., using edge detection) in a region (e.g., region 510) within a threshold distance (e.g., 100 pixel radius) from the initial selection received by via mixed reality interface 112. Refinement component 124 may compare the polynomial curves to the polynomial curve constraints, and in response to detecting correspondence, refinement component 124 may adjust the coordinates of the initial selection to a refined selection.

In some aspects, other image processing techniques such as feature extraction with neural networks and image segmentation may also be used in addition to or instead of traditional image processing techniques. It should be appreciated by one skilled in the art that a variety of image analysis techniques may be utilized to refine virtual fiducial marker placement to coincide more closely with a corresponding physical anatomical landmark.

Still referring to FIG. 8, another aspect of the present disclosure is predicting the placement of virtual fiducial markers on a patient's anatomical landmarks using marker prediction component 126. Using the extended reality device 102's outward-facing cameras and depth sensors as well as image processing techniques (e.g., artificial neural networks trained using supervised learning), an understanding of the scene may be developed such that virtual fiducial marker placement may be proposed by marker prediction component 126 in advance of the user placing a virtual fiducial marker. Use of image processing to generate virtual fiducial marker proposals, especially in an intraoperative context, may serve to expedite placement for the surgeon and reduce the propensity for errors.

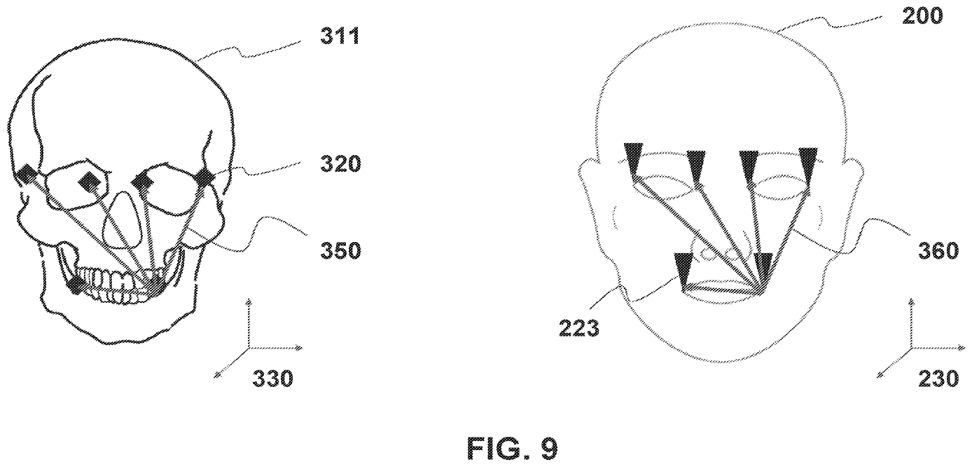

FIG. 9 illustrates a further refinement step of the method to enable accurate virtual fiducial marker placements relative to physical anatomical landmarks. In this step, the relative coordinates of the set of virtual fiducial markers 223 indicated on the physical patient 200 and the relative coordinates of the set of virtual fiducial markers 320 indicated on the 3D virtual model 311 are compared by refinement component 124. Sets of vectors 350 and 360 represent relative translations between sets of vectors corresponding to the coordinates of each set of virtual fiducial markers and may be established by refinement component 124 after all virtual fiducial markers 223 have been indicated on the physical patient. By comparing vectors in sets 350 and 360, refinement component 124 performs a shape analysis that can highlight outliers and either propose refined positions or allow the user to edit the position of one or more virtual fiducial markers. Although sets of vectors 350 and 360 are defined in different coordinate frames 330 and 230, respectively, the scaling of the two sets of vectors is equivalent based on the preoperative segmentation of the 3D models as described herein. Therefore, comparison of these two sets of vectors can provide information on the accuracy of virtual fiducial marker 223 placements compared to the ideal locations corresponding to the patient's physical anatomical landmarks.

By definition, point-set registration techniques consider rotation, translation and scaling of all points in the set. Relative displacements between a subset of the intraoperatively-indicated virtual fiducial markers 223 relative to the predetermined virtual fiducial markers 320 can indicate improper positioning of a subset of virtual fiducial markers 223. Therefore, such a comparison provides a shape analysis that may be used to refine placement by refinement component 124. For example, refinement component 124 may compare two corresponding vectors from sets 350 and 360 (e.g., slope, length, etc.). If a vector from set 360 deviates from the corresponding vector in set 350 by more than a threshold amount, refinement component 124 may determine that the placement of the marker associated with the vector in set 360 needs refinement to bring the difference between the two vectors to below the threshold amount. Accordingly, refinement component 124 may determine a new refined position of the marker that reduces the deviation to below the threshold amount.

In some aspect, as patients undergoing surgery may have swelling and/or localized shape changes due to surgical instrumentation (e.g., swelling from intubation), refinement component 124 may not automatically edit virtual fiducial marker placements but may alert the surgeon, via mixed reality interface 112, to possible deviations and allow the surgeon to make judgments on editing placements. It should be appreciated to one skilled in the art that more sophisticated shape analysis techniques, such as ellipsoid fitting, may be used in addition to simple vector analysis to detect deviations and propose refinements.

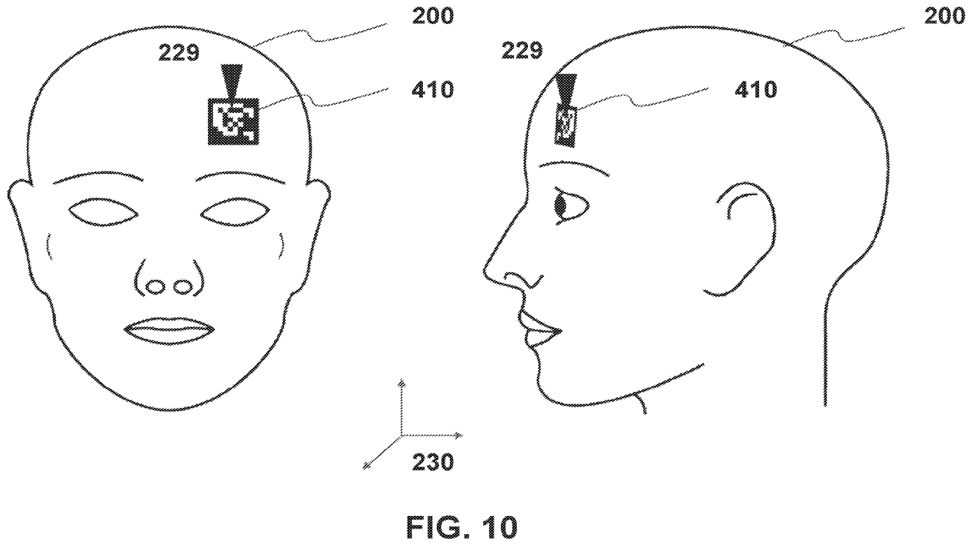

FIG. 10 illustrates how optical codes, such as QR codes or ArUco markers, may be integrated with the virtual fiducial marker registration method. During registration, optical code component 128 may generate optical code 410 and place a virtual fiducial marker 229 on optical code 410 to create correspondence in the coordinate frame 230 between the virtual fiducial markers of the anatomical landmarks and the virtual fiducial marker on the optical code. The optical code 410 may be tracked in real-time by optical code component 128, and used to reorient the entire group of virtual fiducial markers in real-time based on the use of known pose estimation techniques such as SolvePnP. As an optical marker(s), which is rigidly affixed to patient 200, changes its pose relative to the extended reality device 102's camera, the pose of the set of virtual fiducial markers 223 placed by the surgeon may be shifted based on the transformation matrix computed from the updated marker pose, along with any registered 3D models such as 310. This includes if, for example, the patient is to be moved during the process of placing virtual fiducial markers, prior to completion of the registration process. Use of optical markers allows for relative motion to be accurately measured with well-established pose estimation techniques and provides flexibility to the surgeon in placing virtual fiducial markers on the patient.

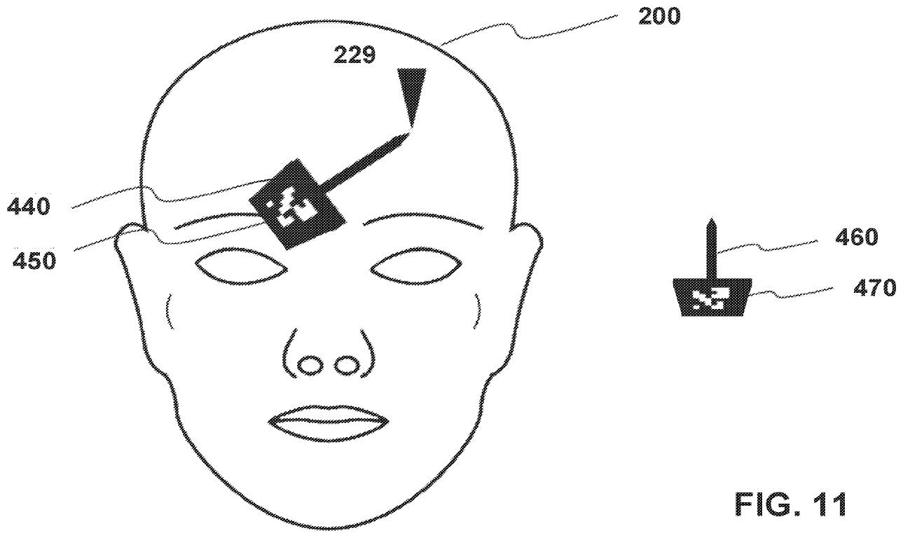

FIG. 11 illustrates how optical codes, such as QR codes or ArUco markers, may be utilized for placement of virtual fiducial markers. A stylus probe 440 of known geometry is held by the surgeon with optical code 450 attached. As described in the preceding paragraph, optical code component 128 applies pose estimation techniques to known optical markers to determine accurate estimations of relative translation and rotation. Because the geometry of stylus probe 440 and optical code 450 is known a priori, the position of the tip of the stylus may be accurately estimated in real-time using optical code component 128. As described previously in the context of virtual ray fiducial marker placements, stylus-based fiducial marker placement can also be commanded when ready by the surgeon through use of a voice command, gesture input, or eye movement.

Still referring to FIG. 11, a second stylus design with corresponding unique marker 470 may be utilized to place virtual fiducial markers. The use of a unique marker identifier informs the application that a different stylus geometry is being used, and allows for virtual fiducial markers to be placed accurately at the tip of second stylus 460 upon command by the surgeon. During a procedure, it may be advantageous to use multiple stylus designs to indicate fiducial markers corresponding to anatomical landmarks. For instance, a sterile landmark such as the tip of a tooth may be used for registration, and it may be preferred to use a marker for a sterile location only once. Alternatively, certain stylus designs may be preferable to access and confidently indicate on certain landmarks.

The designs shown in FIG. 11 and the use of multiple stylus designs in one procedure to indicate a set of virtual fiducial markers are not intended to be limiting in any way. Moreover, the surgeon has the flexibility to use virtual ray indications and stylus indications for a given registration sequence based on preference. The landmark refinement techniques described in the preceding paragraphs, such as mesh contour analysis and shape analysis, may be equivalently applied to refine the position of virtual fiducial markers placed with virtual rays and stylus probes.

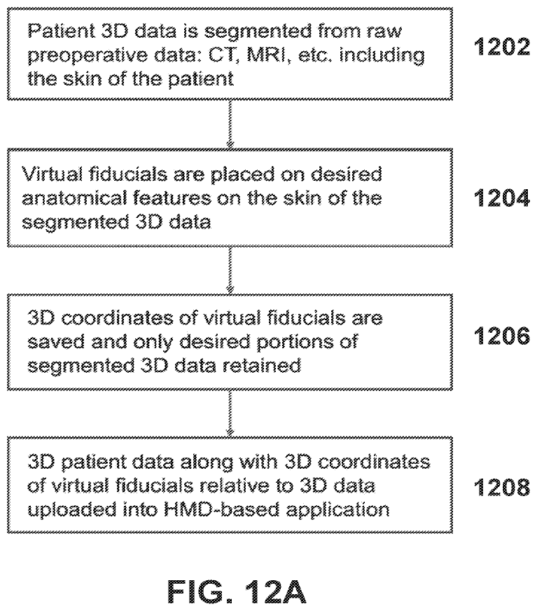

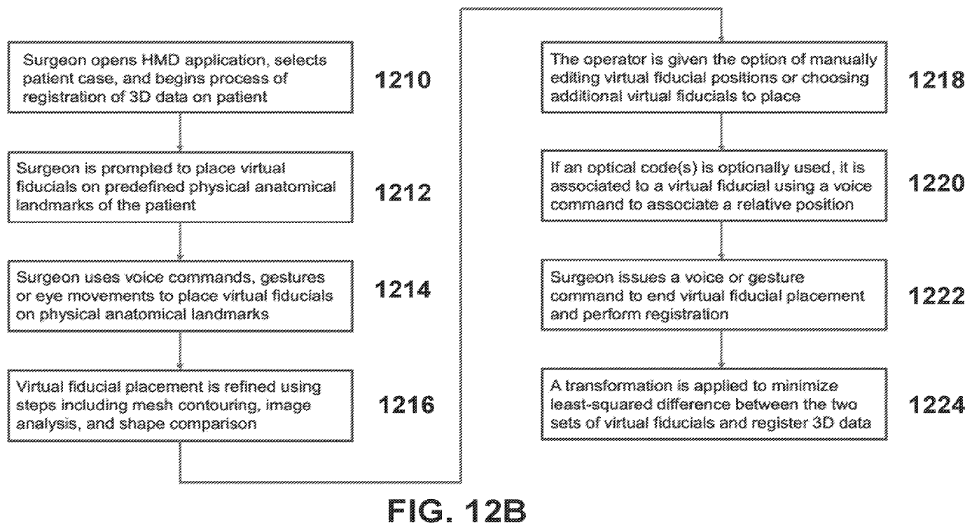

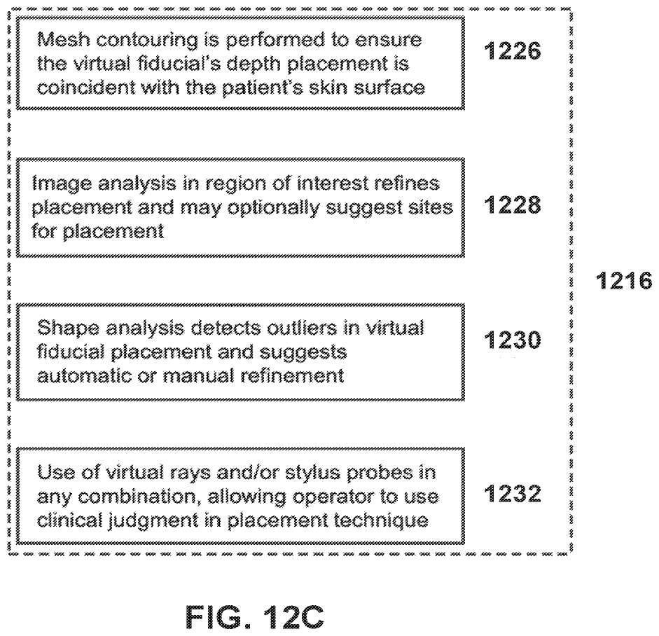

FIG. 12A provides a flowchart of the preoperative steps for placing virtual fiducial markers on the 3D models. In block 1202, patient 3D imaging data is created on a computer (e.g., using open-source software such as 3D Slicer, which converts raw data from a CT scan (in DICOM format) into a 3D mesh(es)). Generally, the region of the scan will envelop beyond the skin of the patient, thereby allowing a mesh to be created of the skin of the patient that may be used as a common reference for virtual fiducial marker placement preoperatively and intraoperatively.

In block 1204, continuing the preoperative virtual fiducial marker placement process, virtual fiducial markers are placed, by a user, on anatomical features of the 3D imaging data that represents the skin of the patient. Registration module 106 allows the user to select points on the surface of the 3D imaging data representing the skin of the patient, according to the planned anatomical landmarks that will be visible to the surgeon at some point during the surgical operation. The placement of these landmarks may be done using standard computing interfaces including but not limited to a keyboard and mouse, and depending on the resolution of the scan data, may allow for sub-millimeter resolution in virtual fiducial marker placement.

Still referring to block 1204, the user may select the position and desired number of anatomical landmarks based on the requirements for that particular surgical case. As illustrated in FIG. 4, virtual fiducial markers may be placed on the 3D imaging data representing the skin of the patient skin on locations such as the corners of the eyes (e.g., the left and right lateral and medial canthus), but may optionally be placed at other locations, such as the nasal tip and subnasale, for example. The choice of landmark locations in this method requires the surgeon to take into account the visibility of the anatomical landmarks during the surgical procedure.

Still referring to block 1204, the number of preoperative virtual fiducial markers placed is dependent on the case. Should surgical draping requirements limit exposure of patient anatomical landmarks, a minimum number of two preoperative virtual fiducial markers may be placed. The preferred minimum number of preoperative virtual fiducial markers is four, which allows for more accurate registration. Ten or more virtual fiducial markers may be placed, but too many fiducial markers may slow down the surgeon when placing virtual fiducial markers intraoperatively. The preferred maximum number of preoperative virtual fiducial markers is six, which provides improved registration accuracy without delaying or distracting the surgeon unnecessarily.

The number of virtual fiducial markers in each set does not need to be equivalent. More preoperative virtual fiducial markers may be placed on the virtual models in this step than will be indicated by the surgeon during surgery. For the method to work, a minimum of four virtual fiducial markers corresponding to physical anatomical landmarks may be placed during the surgery. Ideally, at least six virtual fiducial markers may be placed. If ten virtual fiducial markers are placed on the 3D models during the process step represented by block 1204, and only six are placed intraoperatively on the patient, the method described herein will still function with the accuracy required for the application. The number of preoperative virtual fiducial markers cannot be less than the number placed intraoperatively.

In block 1206, after the virtual fiducial markers are placed, a set of coordinates is exported along with the 3D imaging data, which together are imported into the imaging application on the extended reality device 102 as per block 1208. Optionally, before the 3D imaging data is transferred to the extended reality device 102, some segments of the imaging data may be removed. As a non-limiting example, a CT scan of a head of a patient for a craniotomy surgical case may be segmented into several 3D meshes representing different anatomical structures, including the skin, the skull, the brain, and the jaw. It may be desired for this case to only retain the skull as reference data to register on the patient and view through the extended reality device 102. It should be appreciated by those skilled in the art that once preoperative virtual fiducial markers are placed on the 3D imaging data in a coordinate frame 230, the virtual fiducial markers and all 3D imaging data segments may remain locked in relative position to one another, and therefore removing one segment (such as the skin 3D imaging data) does not affect the relative positions of the preoperative virtual fiducial markers and the remaining 3D imaging data (such as the skull).

FIG. 12B provides a flowchart of the intraoperative steps of placing virtual fiducial markers on the anatomical landmarks of the physical patient, followed by the registration step that results from performing a transformation of the virtual fiducial markers defined preoperatively into the coordinate frame of the virtual fiducial markers defined intraoperatively.

In block 1210, the surgeon accesses registration module 106 on the extended reality device 102 for viewing patient data intraoperatively, and selects the case data corresponding to the 3D imaging data prepared according to the process described by FIG. 12A including the coordinates of the virtual fiducial markers. The surgeon then may initiate a registration process as per block 1212. The surgeon is prompted by registration module 106 to place the intraoperative virtual fiducial markers in a sequence. This sequence may be automatically determined by registration module 106, or may be preoperatively defined by indexing the preoperative virtual fiducial marker coordinates. By way of non-limiting example, the surgeon may first be prompted by registration module 106 to place an intraoperative virtual fiducial marker on the right lateral canthus (corresponding to 212 in FIG. 3), then the right medial canthus (corresponding to 213 in FIG. 3), and progressively place virtual fiducial markers 214, 215, 216, and 217 in no particular order until the predefined number of intraoperative virtual fiducial markers required have been placed.

Referring to blocks 1212, 1214 and 1216 and referencing back to FIG. 6, the placement of intraoperative virtual fiducial markers may be performed by using a gesture-or gaze-based interface of registration module 106 to create a virtual ray 500 with terminus 501 that coincides with the physical surface of the patient 200. Following block 1214 and the non-limiting example referenced above, the surgeon directs virtual ray 500 onto the right lateral canthus of the patient 200, and issues a predefined voice command or gesture command to the extended reality device 102 to indicate the virtual ray terminus 501 is placed in the correct location on the physical patient. As described in FIG. 6 and block 1216, after the surgeon issues the command to place an intraoperative virtual fiducial marker at an indicated location, registration module 106 uses image processing techniques in a small region around the selected point to refine the placement of the virtual fiducial marker and improve its placement accuracy. This process is repeated until all desired intraoperative virtual fiducial markers have been placed.

In block 1218, after the set of intraoperative virtual fiducial markers are placed in their desired locations corresponding to predefined patient anatomical landmarks, the surgeon may optionally refine the locations of the intraoperative virtual fiducial markers. This may be achieved, for example, by using the virtual ray 500 of FIG. 6 to select one of the placed landmarks and issue a voice or gesture command to the extended reality device 102 to enable repositioning following the process of blocks 1212 through 1216 as illustrated in FIG. 6. As a reference to guide the surgeon during editing of virtual fiducial marker placement, identification markers may be used on the virtual fiducial markers, for example a floating text annotation above the virtual fiducial marker indicating the anatomical reference (such as the right lateral canthus).

As per block 1220, if an optical code is optionally used to provide real-time tracking, a virtual fiducial marker is placed by registration module 106 on the code to create a correspondence between the virtual fiducial markers corresponding to physical patient anatomical landmarks and the physical optical code affixed to the patient in the intraoperative coordinate reference frame 230 as illustrated in FIG. 3.

In block 1222, the surgeon issues a voice command or gesture command to end the intraoperative virtual fiducial marker placement process. If a minimum number of virtual fiducial markers are not placed, registration module 106 instructs the surgeon to place the remaining required virtual fiducial markers. Registration module 106 may indicate, to the surgeon, the specific anatomical landmarks that require virtual fiducial markers to be placed. At block 1222, registration module 106 may also perform a verification step to ensure that virtual fiducial markers are spaced out on the physical patient; for example, two or more virtual fiducial markers may not share the same coordinates in the intraoperative coordinate reference frame 230 as illustrated in FIG. 3. Other checks may be performed prior to proceeding to block 1224, such as the orientation of “left” and “right” virtual fiducial markers. The purpose of block 1222 is to lock the placement of the intraoperative virtual fiducial markers and ensure that conditions are probable that block 1224 executes with a successful result.

In block 1224, the transformation of preoperative 3D imaging data with associated preoperative virtual fiducial markers to correspond to the physical patient according to the placement of intraoperative virtual fiducial markers is carried out by registration module 106 using a mathematical relation. Depending on the form of the mathematical relation used to compute the transformation between the two sets of 3D points, a solution may be generated each time even if a poor correspondence between the two sets of points is calculated. Therefore, registration module 106 may be optionally configured to alert the user if the error exceeds a predefined threshold, in effort to warn the user that registration accuracy appears to be poor and advise on remedial actions. Remedial actions may include editing the locations of intraoperative virtual fiducial markers, ensuring virtual fiducial markers were not placed out-of-sequence, and adjusting draping or lighting in the operating theater to ensure better virtual fiducial marker placement.

The result of carrying out process steps in blocks 1202 through 1224 is illustrated in FIG. 5 by item 250, whereby the 3D imaging data of a patient's anatomy is registered to the physical patient. At any time during the procedure, the surgeon may re-initiate this process to re-register the 3D imaging data to the patient. Additionally, through use of an optical code as shown in FIG. 10, or by use of real-time image analytics as performed as part of FIG. 8 to refine virtual fiducial marker locations, the intraoperative virtual fiducial markers may optionally track in real-time any patient movements that occur during the operation, and allow for the 3D imaging data to be re-registered on the patient in real-time. Block 1216 constitutes the critical refinement steps of the method which enable a meaningful advance over the prior art. Block 1216 is detailed from a process flow perspective in the following paragraphs and in FIG. 12C.

Referring now to FIG. 12C, the step represented by block 1216 is expanded to show key elements of the method used to refine the virtual fiducial marker placements made intraoperatively. Block 1226 refers to the mesh contouring approach described by FIG. 7 and used to refine the depth placement of a fiducial marker, whether placed with a virtual ray as shown in FIG. 7, or if placed with a stylus probe as illustrated in FIG. 11. In block 1228, image analysis is performed to refine placement after the surgeon's initial indication, and/or to propose placement prior to indication. Block 1226 and 1228, taken together, help refine the placement of virtual fiducial markers along the three principal coordinate axes, such as coordinate frame 230 in FIG. 8.

Still referring to FIG. 12C, block 1230 represents use of shape analysis to detect outliers in virtual fiducial marker placement. This may be done using a simple vector analysis method as illustrated in FIG. 9, or using more complex curvature and shape analysis methods such as ellipsoid fitting. Block 1230 contributes a verification step to the refinement and may automatically propose position updates to one or more virtual fiducial markers. Alternatively, it may alert the surgeon to outliers and allow them to confirm or deny proposed positional updates based on their clinical judgment. In block 1232, the method allows for the surgeon to place each virtual fiducial marker using a technique that best suits the surgical context. This may involve one or more stylus designs with unique optical markers, along with use of virtual rays to place virtual fiducial markers intraoperatively.

The sub-blocks 1226, 1228, 1230 and 1232 constituting block 1216 of FIG. 12C taken together produce results for intraoperative registration that compare favorably with the most accurate techniques known in the art, such as surgical navigation, without the need for more complex tracking systems and specialized instrumentation. Not all of blocks 1226 to 1232 are required in any given surgical procedure to produce optimal results. It should be appreciated that variations of each of the methods described herein and illustrated in FIGS. 7-11 are anticipated such as the use of more sophisticated shape analysis techniques in block 1230 than illustrated in FIG. 9.

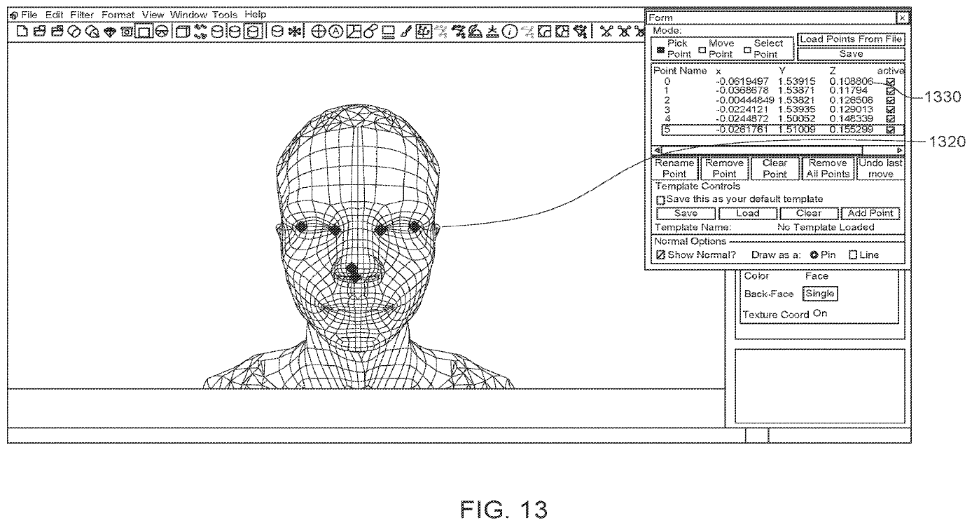

FIG. 13 illustrates an exemplary graphical user interface 114 that enables preoperative placement of virtual fiducial markers. Virtual fiducial markers 1320 are received by registration module 106 using computer inputs (e.g., keyboard, touchpad, mouse, etc.). In some aspects, the GUI 114 may include functionality of software such as MeshLab™, which allows for the preoperative virtual fiducial markers to be selected with high accuracy. Coordinates of each virtual fiducial marker 1320 are generated according to preoperative coordinate reference frame 1330. In some aspects, the GUI 114 of registration module 106 is generated on computing device 104, but intermittently communicates with extended reality device 102 to provide processed 3D models for the purpose of display during surgery.

Still referring to FIG. 13, placement of preoperative virtual fiducial markers 1320 may be performed manually or through automated or semi-automated processes. For instance, a neural network or a set of neural networks may be trained to place preoperative virtual fiducial markers 1320 on the patient or to propose placements to the user of registration module 106. It should be appreciated by one skilled in the art that the aspect described herein is not limiting, and that use of a variety of techniques may be employed to accurately identify virtual fiducial marker placements by registration module 106.

The method described herein has focused on an aspect with one user and extended reality device 102. However, the system and methods of the present disclosure may be practiced with multiple participants and different extended reality device platforms. For instance, virtual fiducial marker placements may be refined by other users simultaneously engaged in a shared experience with their own extended reality devices. Additionally, placements may be guided or refined by a remote proctor, such as an experienced surgeon supervising a surgical resident remotely who is using an extended reality device for guidance in a procedure. Other hardware platforms, such as the Magic Leap 2™, are also compatible with the systems and methods of the present disclosure.

In an exemplary aspect, the extended reality device 102 provides see-through optics with the ability to simultaneously visualize the physical world and three-dimensional digital overlays. The extended reality device is configured to sense and form a mesh or map of its surroundings and includes at least one onboard camera, which may be controlled by software for purposes of image analysis and pose estimation. The extended reality device 102 has sufficient computational resources to manage the tasks described herein. The use of remote servers for real-time inference, rendering, or general computational offloading may also be practiced with the systems and methods of the present disclosure, as not all computational tasks must take place on the extended reality device. Finally, while FIGS. 1-13 primarily describe alignment to the face of a patient, the skin-based registration method described herein generalizes to any part of the anatomy and may be used by a variety of surgical disciplines including orthopedic surgery, thoracic surgery, cardiovascular surgery, general surgery, as well as domains like craniofacial surgery, oral and maxillofacial surgery, otolaryngology, and neurosurgery.

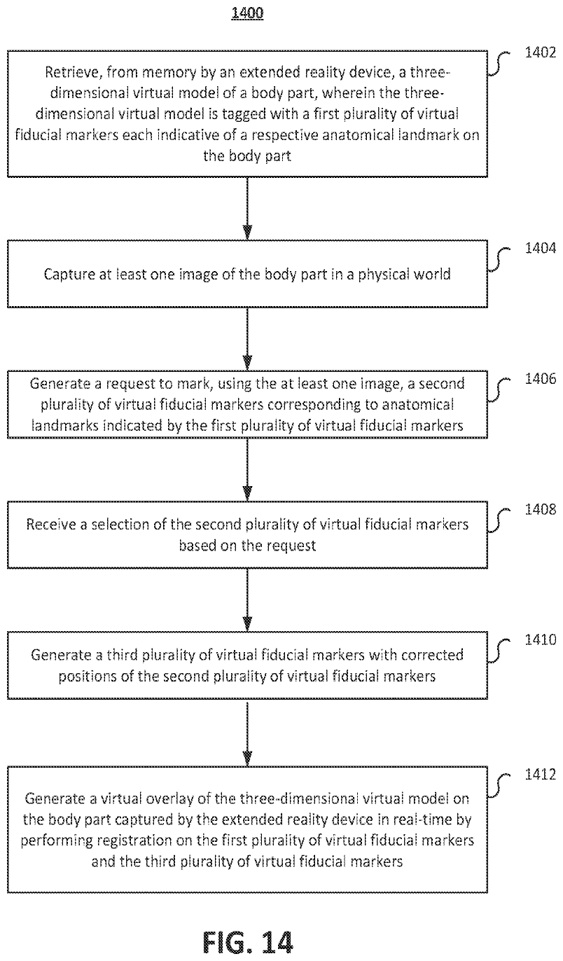

FIG. 14 is a flow diagram illustrating method 1400 for placement of a virtual medical overlay on a physical body part using virtual fiducial markers. At 1402, registration model 106 retrieves, from at least one memory by at least one hardware processor of an extended reality device, a three-dimensional virtual model of a body part. In an exemplary aspect, the three-dimensional virtual model is tagged with a first plurality of virtual fiducial markers each indicative of a respective anatomical landmark on the body part.

In some aspects, the three-dimensional virtual model is generated preoperatively. For example, registration model 106 may generate the three-dimensional virtual model of the body part by performing segmentation on one or more of: X-ray scan, MRI scan, CT scan. Subsequently, registration model 106 may tag the generated three-dimensional virtual model with the first plurality of virtual fiducial markers.

The three-dimensional virtual model with the first plurality of virtual fiducial markers serves as the data that will be overlaid on images of the physical body part being captured by the extended reality device in real-time. In a preferred embodiment, the first plurality of virtual fiducial markers 320 is defined preoperatively using GUI 114 corresponding to locations of anatomical landmarks on preoperative 3D models 300.

At 1404, registration model 106 captures, by the extended reality device, at least one image of the body part in a physical world. For example, registration model 106 may capture a video stream of the physical world and the user may specifically direct the extended reality device at the body part. In some aspects, the extended reality device is worn by a user. The extended reality device may include at least one camera and a display configured to generate virtual reality visuals, augmented reality visuals, or mixed reality visuals.

At 1406, registration model 106 generates a request (e.g., on mixed reality device 112) to mark, using the at least one image, a second plurality of virtual fiducial markers corresponding to anatomical landmarks indicated by the first plurality of virtual fiducial markers. In some aspects, the request comprises a series of requests in which the user is guided through the placement of each respective marker of the second plurality of virtual fiducial markers. In a preferred embodiment, the second plurality of fiducial markers 223 is defined by selecting anatomical landmark locations on a physical patient 200 using mixed reality interface 112.

At 1408, registration model 106 receives a selection of the second plurality of virtual fiducial markers based on the request. In some aspects, the selection is received by registration model 106 using any combination of: (1) tracking a gaze of the user by the at least one camera of the extended reality device; (2) tracking a hand or finger position of the user by the at least one camera; (3) tracking a stylus position by the at least one camera; (4) detecting a voice command by the user; and (5) detecting a physical input on the extended reality device.

In some aspects, the second plurality of virtual fiducial markers comprises at least four markers and at most ten markers. This range of marker count enables accurate registration without slowing down the registration to less than real-time performance. Because surgeries require precise information with minimal time latency, this amount of markers enables superior performance over conventional imaging systems.

At 1410, registration model 106 generates a third plurality of virtual fiducial markers with corrected positions of the second plurality of virtual fiducial markers.

In some aspects, registration model 106 generates the third plurality of virtual fiducial markers by fitting polynomial curves to neighboring features. More specifically, for each respective marker of the second plurality of virtual fiducial markers, registration model 106 identifies, within the at least one image, a region associated with the respective marker. In this case, the respective marker is represented by coordinates and the region is bounded by a threshold distance from the coordinates. Registration model 106 then fits, within the region, at least one polynomial curve on a visual feature captured in the at least one image and retrieves, from the memory, polynomial constraints associated with an anatomical landmark of the respective marker and a relative position of a corrected marker based on the polynomial constraints. Registration model 106 further aligns the respective marker with the corrected marker by comparing the at least one polynomial curve against the polynomial constraints, wherein the aligned respective marker is included in the third plurality of virtual fiducial markers.

In some aspects, registration model 106 generates the third plurality of virtual fiducial markers by using vectors. More specifically, registration model 106 identifies a first anchor marker in the first plurality of virtual fiducial markers and generates a first plurality of vectors originating from the first anchor marker to remaining markers in the first plurality of virtual fiducial markers. Registration model 106 further identifies a second anchor marker in the second plurality of virtual fiducial markers and generates a second plurality of vectors originating from the second anchor marker to remaining markers in the second plurality of virtual fiducial markers. Registration model 106 then repositions each of the second plurality of virtual fiducial markers such that a difference between the first plurality of vectors and the second plurality of vectors is less than a threshold difference, wherein repositioned markers in the second plurality of virtual fiducial markers are included in the third plurality of virtual fiducial markers.

In some aspects, registration model 106 generates the third plurality of virtual fiducial markers using depth information. More specifically, registration model 106 generates a virtual mesh, comprising depth information of the body part, using the at least one image and sensors of the extended reality device. For each respective marker of the second plurality of virtual fiducial markers, registration model 106 determines a terminus point of a virtual ray used to select the respective marker, wherein the terminus point is an intersection of the virtual ray and the virtual mesh. Registration model 106 repositions the respective marker along a depth axis based on the terminus point, wherein the respective marker repositioned is included in the third plurality of virtual fiducial markers.

At 1412, registration model 106 generates a virtual overlay of the three-dimensional virtual model on the body part captured by the extended reality device in real-time by performing registration on the first plurality of virtual fiducial markers and the third plurality of virtual fiducial markers. In some aspects, instead of correcting positions of all the second plurality of virtual fiducial markers, registration model 106 only corrects certain outliers. For example, registration model 106 may perform registration which reduces a difference between corresponding virtual fiducial markers. Suppose that the difference between a majority of the virtual fiducial markers is reduced, but certain markers still maintain a difference. Registration model 106 may detect at least one marker outlier based on a comparison between the first plurality of virtual fiducial markers and the second plurality of virtual fiducial markers and determine a corrected position solely for the at least one marker outlier for inclusion in the third plurality of virtual fiducial markers.

In some aspects, registration model 106 may place a virtual fiducial marker on a physical optical code placed on the body part. This virtual fiducial marker serves as reference point with a trackable position. Whenever the physical optical code moves, the virtual fiducial marker will move as well. Accordingly, registration model 106 updates positions of the second/third plurality of virtual fiducial markers in response to detecting a change in a position of the virtual fiducial marker. If the virtual fiducial marker shifts by X pixels along a particular axis, the plurality of virtual fiducial markers will also be shifted proportionally to X.

In some aspects, registration model 106 may predict positions of each of the third plurality of virtual fiducial markers using at least one machine learning model trained to output the positions based on an input comprising the at least one image and the three-dimensional virtual model.

FIG. 15 is a block diagram illustrating a computer system 20 on which aspects of systems and methods for placement of a virtual medical overlay on a physical body part using virtual fiducial markers may be implemented in accordance with an exemplary aspect. The computer system 20 may be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices. For example, computer system 20 may be computing device 104 and/or may be part of extended reality device 102.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I²C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in FIGS. 1-14 may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which may be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium may be a tangible device that can retain and store program code in the form of instructions or data structures that may be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein may be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some aspects, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure may be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The systems and methods of the present disclosure encompass the following clauses.

Clause 1. A method for placement of a virtual medical overlay on a physical body part using virtual fiducial markers, the method comprising: retrieving, from memory by an extended reality device, a three-dimensional virtual model of a body part, wherein the three-dimensional virtual model is tagged with a first plurality of virtual fiducial markers each indicative of a respective anatomical landmark on the body part; capturing, by the extended reality device, at least one image of the body part in a physical world; generating a request to mark, using the at least one image, a second plurality of virtual fiducial markers corresponding to anatomical landmarks indicated by the first plurality of virtual fiducial markers; in response to receiving a selection of the second plurality of virtual fiducial markers based on the request, generating a third plurality of virtual fiducial markers with corrected positions of the second plurality of virtual fiducial markers; and generating a virtual overlay of the three-dimensional virtual model on the body part captured by the extended reality device in real-time by performing registration on the first plurality of virtual fiducial markers and the third plurality of virtual fiducial markers.

Clause 2. The method of any one or more preceding clauses, wherein generating the third plurality of virtual fiducial markers comprises, for each respective marker of the second plurality of virtual fiducial markers: identifying, within the at least one image, a region associated with the respective marker, wherein the respective marker is represented by coordinates and the region is bounded by a threshold distance from the coordinates; fitting, within the region, at least one polynomial curve on a visual feature captured in the at least one image; retrieving, from the memory, polynomial constraints associated with an anatomical landmark of the respective marker and a relative position of a corrected marker based on the polynomial constraints; and aligning the respective marker with the corrected marker by comparing the at least one polynomial curve against the polynomial constraints, wherein the aligned respective marker is included in the third plurality of virtual fiducial markers.

Clause 3. The method of any one or more of the preceding clauses, wherein generating the third plurality of virtual fiducial markers comprises: identifying a first anchor marker in the first plurality of virtual fiducial markers; generating a first plurality of vectors originating from the first anchor marker to remaining markers in the first plurality of virtual fiducial markers; identifying a second anchor marker in the second plurality of virtual fiducial markers; generating a second plurality of vectors originating from the second anchor marker to remaining markers in the second plurality of virtual fiducial markers; and repositioning each of the second plurality of virtual fiducial markers such that a difference between the first plurality of vectors and the second plurality of vectors is less than a threshold difference, wherein repositioned markers in the second plurality of virtual fiducial markers are included in the third plurality of virtual fiducial markers.

Clause 4. The method of any one or more of the preceding clauses, wherein generating the third plurality of virtual fiducial markers comprises: generating a virtual mesh of body part using the at least one image and sensors of the extended reality device, wherein the virtual mesh comprises depth information of the body part; and for each respective marker of the second plurality of virtual fiducial markers: determining a terminus point of a virtual ray used to select the respective marker, wherein the terminus point is an intersection of the virtual ray and the virtual mesh; and repositioning the respective marker along a depth axis based on the terminus point, wherein the respective marker repositioned is included in the third plurality of virtual fiducial markers.

Clause 5. The method of any one or more of the preceding clauses, further comprising: generating the three-dimensional virtual model of the body part by performing segmentation on one or more of: X-ray scan, MRI scan, CT scan, an ultrasound scan.

Clause 6. The method of any one or more of the preceding clauses, further comprising: tagging the generated three-dimensional virtual model with the first plurality of virtual fiducial markers.

Clause 7. The method of any one or more of the preceding clauses, wherein the second plurality of virtual fiducial markers comprises at least four markers and at most ten markers.

Clause 8. The method of any one or more of the preceding clauses, further comprising: placing a virtual fiducial marker on a physical optical code placed on the body part; and updating positions of the third plurality of virtual fiducial markers in response to detecting a change in a position of the virtual fiducial marker, wherein the virtual fiducial marker is a reference point with a trackable position.

Clause 9. The method of any one or more of the preceding clauses, wherein the extended reality device is worn by a user, and wherein the extended reality device comprises at least one camera and a display configured to generate virtual reality visuals, augmented reality visuals, or mixed reality visuals.

Clause 10. The method of any one or more of the preceding clauses, wherein the selection is received by any combination of: (1) tracking a gaze of the user by the at least one camera; (2) tracking a hand or finger position of the user by the at least one camera; (3) tracking a stylus position by the at least one camera; (4) detecting a voice command by the user; and (5) detecting a physical input on the extended reality device.

Clause 11. The method of any one or more of the preceding clauses, further comprising predicting positions of each of the third plurality of virtual fiducial markers using at least one machine learning model trained to output the positions based on an input comprising the at least one image and the three-dimensional virtual model.

Clause 12. The method of any one or more of the preceding clauses, further comprising: detecting at least one marker outlier based on a comparison between the first plurality of virtual fiducial markers and the second plurality of virtual fiducial markers; and determining a corrected position solely for the at least one marker outlier for inclusion in the third plurality of virtual fiducial markers.

Clause 13. A system for placement of a virtual medical overlay on a physical body part using virtual fiducial markers, comprising: at least one memory; and at least one hardware processor of an extended reality device, wherein the at least one hardware processor is coupled with the at least one memory and configured, individually or in combination, to: retrieve, from the at least one memory, a three-dimensional virtual model of a body part, wherein the three-dimensional virtual model is tagged with a first plurality of virtual fiducial markers each indicative of a respective anatomical landmark on the body part; capture at least one image of the body part in a physical world; generate a request to mark, using the at least one image, a second plurality of virtual fiducial markers corresponding to anatomical landmarks indicated by the first plurality of virtual fiducial markers; in response to receiving a selection of the second plurality of virtual fiducial markers based on the request, generate a third plurality of virtual fiducial markers with corrected positions of the second plurality of virtual fiducial markers; and generate a virtual overlay of the three-dimensional virtual model on the body part captured by the extended reality device in real-time by performing registration on the first plurality of virtual fiducial markers and the third plurality of virtual fiducial markers.

Clause 14. The system of any one or more of the preceding clauses, wherein the at least one hardware processor is configured to generate the third plurality of virtual fiducial markers by, for each respective marker of the second plurality of virtual fiducial markers: identifying, within the at least one image, a region associated with the respective marker, wherein the respective marker is represented by coordinates and the region is bounded by a threshold distance from the coordinates; fitting, within the region, at least one polynomial curve on a visual feature captured in the at least one image; retrieving, from the memory, polynomial constraints associated with an anatomical landmark of the respective marker and a relative position of a corrected marker based on the polynomial constraints; and aligning the respective marker with the corrected marker by comparing the at least one polynomial curve against the polynomial constraints, wherein the aligned respective marker is included in the third plurality of virtual fiducial markers.

Clause 15. The system of any one or more of the preceding clauses, wherein the at least one hardware processor is configured to generate the third plurality of virtual fiducial markers by: identifying a first anchor marker in the first plurality of virtual fiducial markers; generating a first plurality of vectors originating from the first anchor marker to remaining markers in the first plurality of virtual fiducial markers; identifying a second anchor marker in the second plurality of virtual fiducial markers; generating a second plurality of vectors originating from the second anchor marker to remaining markers in the second plurality of virtual fiducial markers; and repositioning each of the second plurality of virtual fiducial markers such that a difference between the first plurality of vectors and the second plurality of vectors is less than a threshold difference, wherein repositioned markers in the second plurality of virtual fiducial markers are included in the third plurality of virtual fiducial markers.

Clause 16. The system of any one or more of the preceding clauses, wherein the at least one hardware processor is configured to generate the third plurality of virtual fiducial markers by: generating a virtual mesh of body part using the at least one image and sensors of the extended reality device, wherein the virtual mesh comprises depth information of the body part; and for each respective marker of the second plurality of virtual fiducial markers: determining a terminus point of a virtual ray used to select the respective marker, wherein the terminus point is an intersection of the virtual ray and the virtual mesh; and repositioning the respective marker along a depth axis based on the terminus point, wherein the respective marker repositioned is included in the third plurality of virtual fiducial markers.

Clause 17. The system of any one or more of the preceding clauses, wherein the at least one hardware processor is configured to: generate the three-dimensional virtual model of the body part by performing segmentation on one or more of: an X-ray scan, an MRI scan, an ultrasound scan, and/or a CT scan.

Clause 18. The system of any one or more of the preceding clauses, wherein the at least one hardware processor is configured to: tag the generated three-dimensional virtual model with the first plurality of virtual fiducial markers.

Clause 19. The system of any one or more of the preceding clauses, wherein the second plurality of virtual fiducial markers comprises at least four markers and at most ten markers.

Clause 20. A non-transitory computer readable medium storing thereon computer executable instructions for placement of a virtual medical overlay on a physical body part using virtual fiducial markers, including instructions for: retrieving, from memory by an extended reality device, a three-dimensional virtual model of a body part, wherein the three-dimensional virtual model is tagged with a first plurality of virtual fiducial markers each indicative of a respective anatomical landmark on the body part; capturing, by the extended reality device, at least one image of the body part in a physical world; generating a request to mark, using the at least one image, a second plurality of virtual fiducial markers corresponding to anatomical landmarks indicated by the first plurality of virtual fiducial markers; in response to receiving a selection of the second plurality of virtual fiducial markers based on the request, generating a third plurality of virtual fiducial markers with corrected positions of the second plurality of virtual fiducial markers; and generating a virtual overlay of the three-dimensional virtual model on the body part captured by the extended reality device in real-time by performing registration on the first plurality of virtual fiducial markers and the third plurality of virtual fiducial markers.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

1. A method for placement of a virtual medical overlay on a physical body part using virtual fiducial markers, the method comprising:
retrieving, from memory by an extended reality device, a three-dimensional virtual model of a body part, wherein the three-dimensional virtual model is tagged with a first plurality of virtual fiducial markers each indicative of a respective anatomical landmark on the body part;
capturing, by the extended reality device, at least one image of the body part in a physical world;
generating a request to mark, using the at least one image, a second plurality of virtual fiducial markers corresponding to anatomical landmarks indicated by the first plurality of virtual fiducial markers;
in response to receiving a selection of the second plurality of virtual fiducial markers based on the request, generating a third plurality of virtual fiducial markers with corrected positions of the second plurality of virtual fiducial markers; and
generating a virtual overlay of the three-dimensional virtual model on the body part captured by the extended reality device in real-time by performing registration on the first plurality of virtual fiducial markers and the third plurality of virtual fiducial markers.

2. The method of claim 1, wherein generating the third plurality of virtual fiducial markers comprises, for each respective marker of the second plurality of virtual fiducial markers:
identifying, within the at least one image, a region associated with the respective marker, wherein the respective marker is represented by coordinates and the region is bounded by a threshold distance from the coordinates;
fitting, within the region, at least one polynomial curve on a visual feature captured in the at least one image;
retrieving, from the memory, polynomial constraints associated with an anatomical landmark of the respective marker and a relative position of a corrected marker based on the polynomial constraints; and
aligning the respective marker with the corrected marker by comparing the at least one polynomial curve against the polynomial constraints, wherein the aligned respective marker is included in the third plurality of virtual fiducial markers.

3. The method of claim 1, wherein generating the third plurality of virtual fiducial markers comprises:
identifying a first anchor marker in the first plurality of virtual fiducial markers;
generating a first plurality of vectors originating from the first anchor marker to remaining markers in the first plurality of virtual fiducial markers;
identifying a second anchor marker in the second plurality of virtual fiducial markers;
generating a second plurality of vectors originating from the second anchor marker to remaining markers in the second plurality of virtual fiducial markers; and
repositioning each of the second plurality of virtual fiducial markers such that a difference between the first plurality of vectors and the second plurality of vectors is less than a threshold difference, wherein repositioned markers in the second plurality of virtual fiducial markers are included in the third plurality of virtual fiducial markers.

4. The method of claim 1, wherein generating the third plurality of virtual fiducial markers comprises:
generating a virtual mesh of body part using the at least one image and sensors of the extended reality device, wherein the virtual mesh comprises depth information of the body part; and
for each respective marker of the second plurality of virtual fiducial markers:
determining a terminus point of a virtual ray used to select the respective marker, wherein the terminus point is an intersection of the virtual ray and the virtual mesh; and
repositioning the respective marker along a depth axis based on the terminus point, wherein the respective marker repositioned is included in the third plurality of virtual fiducial markers.

5. The method of claim 1, further comprising:
generating the three-dimensional virtual model of the body part by performing segmentation on one or more of: X-ray scan, MRI scan, CT scan, an ultrasound scan.

6. The method of claim 5, further comprising:
tagging the generated three-dimensional virtual model with the first plurality of virtual fiducial markers.

7. The method of claim 1, wherein the second plurality of virtual fiducial markers comprises at least four markers and at most ten markers.

8. The method of claim 1, further comprising:
placing a virtual fiducial marker on a physical optical code placed on the body part; and
updating positions of the third plurality of virtual fiducial markers in response to detecting a change in a position of the virtual fiducial marker, wherein the virtual fiducial marker is a reference point with a trackable position.

9. The method of claim 1, wherein the extended reality device is worn by a user, and wherein the extended reality device comprises at least one camera and a display configured to generate virtual reality visuals, augmented reality visuals, or mixed reality visuals.

10. The method of claim 9, wherein the selection is received by any combination of:
(1) tracking a gaze of the user by the at least one camera;
(2) tracking a hand or finger position of the user by the at least one camera;
(3) tracking a stylus position by the at least one camera;
(4) detecting a voice command by the user; and
(5) detecting a physical input on the extended reality device.

11. The method of claim 1, further comprising predicting positions of each of the third plurality of virtual fiducial markers using at least one machine learning model trained to output the positions based on an input comprising the at least one image and the three-dimensional virtual model.

12. The method of claim 1, further comprising:
detecting at least one marker outlier based on a comparison between the first plurality of virtual fiducial markers and the second plurality of virtual fiducial markers; and
determining a corrected position solely for the at least one marker outlier for inclusion in the third plurality of virtual fiducial markers.

13. A system for placement of a virtual medical overlay on a physical body part using virtual fiducial markers, comprising:
at least one memory; and
at least one hardware processor of an extended reality device, wherein the at least one hardware processor is coupled with the at least one memory and configured, individually or in combination, to:
retrieve, from the at least one memory, a three-dimensional virtual model of a body part, wherein the three-dimensional virtual model is tagged with a first plurality of virtual fiducial markers each indicative of a respective anatomical landmark on the body part;
capture at least one image of the body part in a physical world;
generate a request to mark, using the at least one image, a second plurality of virtual fiducial markers corresponding to anatomical landmarks indicated by the first plurality of virtual fiducial markers;
in response to receiving a selection of the second plurality of virtual fiducial markers based on the request, generate a third plurality of virtual fiducial markers with corrected positions of the second plurality of virtual fiducial markers; and
generate a virtual overlay of the three-dimensional virtual model on the body part captured by the extended reality device in real-time by performing registration on the first plurality of virtual fiducial markers and the third plurality of virtual fiducial markers.

14. The system of claim 13, wherein the at least one hardware processor is configured to generate the third plurality of virtual fiducial markers by, for each respective marker of the second plurality of virtual fiducial markers:
identifying, within the at least one image, a region associated with the respective marker, wherein the respective marker is represented by coordinates and the region is bounded by a threshold distance from the coordinates;
fitting, within the region, at least one polynomial curve on a visual feature captured in the at least one image;
retrieving, from the memory, polynomial constraints associated with an anatomical landmark of the respective marker and a relative position of a corrected marker based on the polynomial constraints; and
aligning the respective marker with the corrected marker by comparing the at least one polynomial curve against the polynomial constraints, wherein the aligned respective marker is included in the third plurality of virtual fiducial markers.

15. The system of claim 13, wherein the at least one hardware processor is configured to generate the third plurality of virtual fiducial markers by:
identifying a first anchor marker in the first plurality of virtual fiducial markers;
generating a first plurality of vectors originating from the first anchor marker to remaining markers in the first plurality of virtual fiducial markers;
identifying a second anchor marker in the second plurality of virtual fiducial markers;
generating a second plurality of vectors originating from the second anchor marker to remaining markers in the second plurality of virtual fiducial markers; and
repositioning each of the second plurality of virtual fiducial markers such that a difference between the first plurality of vectors and the second plurality of vectors is less than a threshold difference, wherein repositioned markers in the second plurality of virtual fiducial markers are included in the third plurality of virtual fiducial markers.

16. The system of claim 13, wherein the at least one hardware processor is configured to generate the third plurality of virtual fiducial markers by:
generating a virtual mesh of body part using the at least one image and sensors of the extended reality device, wherein the virtual mesh comprises depth information of the body part; and
for each respective marker of the second plurality of virtual fiducial markers:
determining a terminus point of a virtual ray used to select the respective marker, wherein the terminus point is an intersection of the virtual ray and the virtual mesh; and
repositioning the respective marker along a depth axis based on the terminus point, wherein the respective marker repositioned is included in the third plurality of virtual fiducial markers.

17. The system of claim 13, wherein the at least one hardware processor is configured to:
generate the three-dimensional virtual model of the body part by performing segmentation on one or more of: an X-ray scan, an MRI scan, an ultrasound scan, and/or a CT scan.

18. The system of claim 17, wherein the at least one hardware processor is configured to:
tag the generated three-dimensional virtual model with the first plurality of virtual fiducial markers.

19. The system of claim 13, wherein the second plurality of virtual fiducial markers comprises at least four markers and at most ten markers.

20. A non-transitory computer readable medium storing thereon computer executable instructions for placement of a virtual medical overlay on a physical body part using virtual fiducial markers, including instructions for:
retrieving, from memory by an extended reality device, a three-dimensional virtual model of a body part, wherein the three-dimensional virtual model is tagged with a first plurality of virtual fiducial markers each indicative of a respective anatomical landmark on the body part;
capturing, by the extended reality device, at least one image of the body part in a physical world;
generating a request to mark, using the at least one image, a second plurality of virtual fiducial markers corresponding to anatomical landmarks indicated by the first plurality of virtual fiducial markers;
in response to receiving a selection of the second plurality of virtual fiducial markers based on the request, generating a third plurality of virtual fiducial markers with corrected positions of the second plurality of virtual fiducial markers; and
generating a virtual overlay of the three-dimensional virtual model on the body part captured by the extended reality device in real-time by performing registration on the first plurality of virtual fiducial markers and the third plurality of virtual fiducial markers.
